(19) United States
(12) Patent Application Publication
(10) Pub. No.: US 2006/0100438 A1
(43) Pub. Date: May 11, 2006

(54) Process of making fentanyl intermediates

(75) Inventor: Mark Richard Rubino, Midlothian, VA (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Chemicals, Inc., Petersburg, VA (US)

(21) Appl. No.: 11/268,241

(22) Filed: Nov. 7, 2005

Related U.S. Application Data
(60) Provisional application No. 60/626,692, filed on Nov. 10, 2004.

(51) Int. Cl. C07D 211/56

(52) U.S. Cl. 546/223

(57) ABSTRACT

Disclosed are processes of making the fentanyl intermediate 1-(2-phenethyl)-4-anilinopiperidine. Also disclosed are methods of isolating the compound.

This application claims benefit to U.S. provisional application No. 60/626,692 filed Nov. 10, 2004.

### BACKGROUND OF THE INVENTION

1. Technical Field

This invention relates a process of making intermediates useful in the synthesis of fentanyl, a drug substance extensively used for anesthesia and analgesia.

2. Background Information

Fentanyl has an analgesic potency of about 80 times that of morphine. Fentanyl and its salts are extensively used for anesthesia and analgesia. There are a several dosing options for fentanyl, including the Duragesic® transdermal patch used in chronic pain management, and the Actiq® solid formulation of fentanyl on a stick that dissolves slowly in the mouth for transmucosal absorption for treatment of breakthrough pain in cancer patients. Several new fentanyl drug products are in development and clinical trials—these products offer increased flexibility and patient convenience in dosing for management of chronic and breakthrough pain.

A well known process for synthesizing fentanyl and the intermediates thereof is disclosed in Przemysl Chemiczny 1978, 57, 4, 180-182 by A. Jonczyk, M. Jawdosiuk and M. Makosza (in Polish language). Many known manufacturing processes are based on the Jonczyk process for making 1-(2-phenethyl)-4-anilinopiperidine.

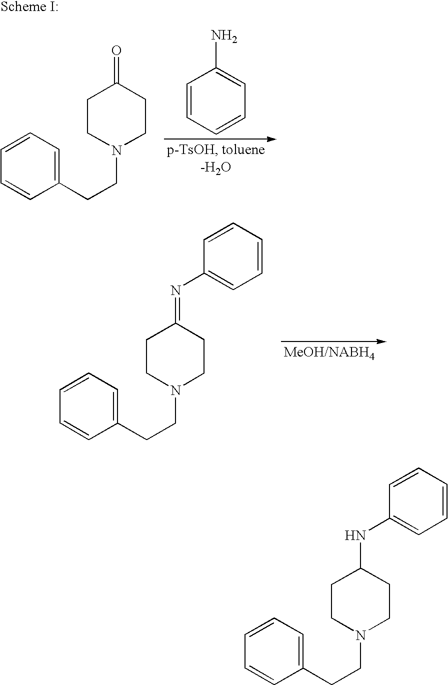

The publication reports two examples: an overall process yield of 73% for 1-(2-phenethyl)-4-anilinopiperidine recrystallized from cyclohexane and an overall process yield of 66% when the product is recrystallized from methanol. As shown in scheme I, known processes for 1 -phenethyl-4-phenylaminopiperidine require a discrete imine formation from 1-phenethyl-4-piperidone and aniline with acid catalysis, followed by imine reduction in methanolic sodium borohydride solution. Utilizing these conditions has required not only monitoring of the formation of the imine, but also the reduction to the desired diamine. All this reaction monitoring and the instability of the imine has resulted in the need for an improved process. A chemical alternative to dealing with known two step processes would be the use of reductive amination conditions. These types of processes are well described in the literature and are used industrially. E. W. Baxter and A. B. Reitz “Reductive Aminations of Carbonyl Compounds with Borohydride and Borane Reducing Agents” Organic Reactions, Editor: L. E. Overman, 2002, Volume 59. Most commonly catalytic hydrogenation is used in industry but dedicated equipment is required to handle hydrogen and active catalysts. Skita, A. Keil, F. Chem. Ber. 1928, 61B,1452. Other methods rely on expensive reagents or reagents that demand special handling. Hutchins, R. O. Hutchins, M. K. Reduction of C═N to CHNH by Metal Hydrides. In Comprehensive Organic Synthesis; Editors B. N. Trost and I. Fleming, 1991, Volume 8.

U.S. Pat. Nos. 6,051,717 and 6,689,913 disclose reductive alkylation which is performed using sodium triacetoxyborohydride in toluene. The substances produced are other than 1-(2-phenethyl)-4-anilinopiperidine.

The preparation of sodium triacetoxyborohydride in benzene is described in The Journal of the American Chemical Society 1988, 110, 3560-3578 by D. A. Evans, K. T. Chapman and E. M. Carriera “Directed Reduction of β-Hydroxy Ketones Employing Tetramethylammonium Triacetoxyborohydride”.

### BRIEF SUMMARY OF THE INVENTION

The work cited above supports the principle that an improved method for producing 1-(2-phenethyl)-4-anilinopiperidine useful for making fentanyl and the salts thereof is desirable.

It is therefore an object of the invention to provide a simpler, more efficient process of preparing 1-(2-phenethyl)-4-anilinopiperidine.

It is another object of the invention to provide a process of purifying 1-(2-phenethyl)-4-anilinopiperidine.

### DETAILED DESCRIPTION OF THE INVENTION

The present invention provides for a synthesis of 1-(2-phenethyl)-4-anilinopiperidine (PAP).

The present invention shown in Scheme II below solves the process issues of known processes of making PAP by the use of a reductive amination with a modified borohydride reagent. One critical difference in using a reductive amination versus a distinctive two step amination is the reagent used in the reduction. Reductive amination according to the present invention is done with sodium triacetoxyborohydride (NaBH(OAc)₃) instead of sodium borohydride and the sodium triacetoxyborohydride can be generated from sodium borohydride and acetic acid.

In one aspect of the invention, there is provided a process of preparing 1-(2-phenethyl)-4-anilinopiperidine in good yield, said process comprising:

reacting 1-(2-phenethyl)-4-piperidone with aniline in a suitable solvent, the suitable solvent including aromatic solvents, preferably six to nine carbon aromatic solvents, non-limiting examples are: xylene, cumene, ethylbenzene, benzene, trimethyl benzene ethyl toluene, cymene, and toluene and the mixtures thereof and all isomers thereof; more preferably the solvent is toluene optionally with an acid;

suitable acids include formic, acetic, propionic, butyric, valeric, hexanoic, isobutyric, isovaleric, pivalic, chloroacetic, methoxyacetic, benzoic, phthalic, isophthalic, picolinic, nicotinic, isonicotinic, 2-ethylhexanoic, toluic, glycolic, phenylacetic, citric, citramalic, anisic, fumaric, malic, oxalic, malonic, glutaric, maleic, cyclohexanecarboxylic, 2-methylbutyric, tartaric, ascorbic, sorbic, salicylic, p-hydroxybenzoic, 2-propylheptanoic, trimethyladipic, mandelic, (4-isobutylphenyl)propionic, furoic, diphenolic, lactic, 2-methylglutaric, 2,2-dimethylmalonic, 3-hydroxy-2,2-dimethylpropionic, phenoxyacetic, acetylsalicylic, N-acetyl-DL-methionine, N-acetyl-L-glutamic acid, 6-acetamidohexanoic;

preferred acids include acetic, propionic, butyric, valeric, hexanoic, isobutyric, isovaleric, pivalic, 2-methylbutyric, benzoic;

most preferred is acetic acid;

adding sodium triacetoxyborohydride, or optionally sodium triacyloxyborohydride generated in-situ with acid and sodium borohydride (preferably sodium triacetoxyborohydride generated in-situ with acetic acid and sodium borohydride) to produce 1-(2-phenethyl)-4-anilinopiperidine;

isolating 1-(2-phenethyl)-4-anilinopiperidine.

It shall be understood that the invention is not limited to the order of steps recited above. The process works with a variety of addition sequences, for example: sodium triacetoxyborohydride in toluene can be stirred while all other components are added as a solution (or slurry) in toluene.

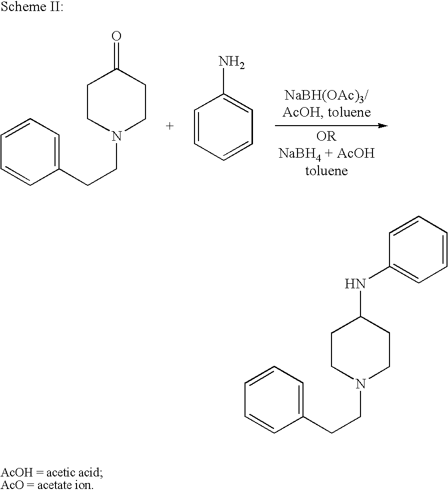

In an additional aspect of the invention, there is provided an improved purification process of 1-(2-phenethyl)-4-anilinopiperidine by recrystallization from a suitable solvent chosen from 1-propanol, 2-butanol, methyl isopropyl ketone, 2,2-dimethyl-1,3-dioxolane, tetrahydrofuran, 2-Methyltetrahydrofuran, tetrahydropyran, 1,2-dimethoxyethane, 2-butanone, pyrrolidine, 1-Methylpyrrolidine, triethylamine, diisopropylamine, acetonitrile, 2,2-dimethoxypropane, diethoxymethane, 1,3-dioxolane, 2-methyl-1,3-dioxolane ethyl acetate, isopropyl acetate, ethyl isobutyrate and 2-propanol said process comprising,

providing 1-(2-phenethyl)-4-anilinopiperidine in the solvent;

isolating the 1-(2-phenethyl)-4-anilinopiperidine product.

The corresponding yield according to the present invention using a preferred solvent, 2-propanol, is 87.6% yield in the single example. The 87.6% yield is not the upper limit. All the other examples in the table below are for 1-(2-phenethyl)-4-anilinopiperidine without recrystallization.

Reductive amination procedures are known, such as that described in the Journal of Organic Chemistry, 1996, 61, 3849-3862 by A. F. Abdel-Magid, K. G. Carson, B. D. Harris, C. A. Maryanoff and R. D. Shah “Reductive Amination of Aldehydes and Ketones with Sodium Triacetoxyborohydride”. The process of the invention uses sodium triacetoxyborohydride (commercially available) as well as with sodium triacetoxyborohydride made in toluene by adding acetic acid to sodium borohydride.

1-(2-phenethyl)-4-anilinopiperidine is an intermediate in the manufacturing process for fentanyl hydrochloride, base and citrate. To produce this intermediate, known processes use raw materials of aniline, toluene and 1-(2-phenethyl)-4-piperidone as shown in the background section above. Surprisingly, it has been found by the present inventors that uses of sodium triacetoxyborohydride, and/or optionally sodium triacetoxyborohydride generated in-situ with acetic acid and sodium borohydride, with the aforementioned raw materials provides a shorter, simpler and higher yield than the processes known in the art.

The improved purification process of 1-(2-phenethyl)-4-anilinopiperidine by recrystallization includes use of a single solvent. The use of a single solvent according to the present invention provides a simpler and more efficient purification. The melting point of 1-(2-phenethyl)-4-anilinopiperidine is about 96° C. and so a recrystallization solvent with boiling point close to or slightly higher can be used, such solvents include 1-propanol, 2-butanol, methyl isopropyl ketone, 2,2-dimethyl-1,3-dioxolane. Preferred are solvents with a boiling point somewhat below 96° C., such solvents include tetrahydrofuran, 2-Methyltetrahydrofuran, tetrahydropyran, 1,2-dimethoxyethane, 2-butanone, pyrrolidine, 1-Methylpyrrolidine, triethylamine, diethoxymethane, 1,3-dioxolane, 2-methyl-1,3-dioxolane and 2-propanol. The solvent most preferred is 2-propanol, in part due to the higher boiling point of 2-propanol (82.5° C.) versus methanol (64.7° C.).

In order that this invention be more fully understood, the following examples are set forth. These examples are for the purpose of illustrating preferred embodiments of this invention, and are not to be construed as limiting the scope of the invention in any way.

The experiments shown in the table below using Aldrich (powdered) NaBH(OAc)₃ were based on the general method described in Abdel-Magid et al. Toluene was used rather than the suggested 1,2-dichloroethane. The result was both high yield and high conversion with either purified or crude phenethyl piperidone. The operation can be carried out on lab scale without special monitoring. Conversely the reagent can be made from NaBH₄ and acetic acid and used under standard conditions.

It is also within the scope of the invention to substitute aniline for some or all of the aniline—acetic acid salt. The stoichiometry for the process may be varied as will be appreciated by those skilled in the art, such use of reduced amounts of NaBH(OAc)₃. Shorter reaction times are possible since long reaction times were intentionally used to insure full conversion. The completion of reaction was readily determined with either gas chromatography or high pressure liquid chromatography or thin layer chromatography.

During experiment 4 colorless crystals formed in the toluene layer while standing over the basic aqueous layer. The crystals were collected and found to be PAP containing substantial levels of water (4% to 7%) that was otherwise pure. These crystals may contain PAP hydrate. This substance does not appear in the literature and the crystallization may show that PAP hydrate has reduced solubility in toluene relative to PAP itself.

### EXAMPLE

### Synthesis of 1-phenethyl-4-phenylaminopiperidine (PAP) using sodium triacetoxyborohydride

Procedure: To a flask, solid 1-(2-phenethyl)-4-piperidone (“phenethyl piperidone”) (64.27 g, F.W. 203.28, 0.31616 mole), liquid aniline (29.79 g, F.W. 93.13, 0.3199 mole), liquid acetic acid (19.15 g, F.W. 60.05, 0.3189 mole) and liquid toluene (340 g) was added. The mixture was agitated under a slight positive pressure of nitrogen and heated to a solution temperature of 50-60° C. and held in this range for fifteen (15) minutes to dissolve all the phenethyl piperidone. The reaction flask was then cooled to a solution temperature of 0±5° C.

Powdered sodium triacetoxyborohydride (94.26 g, F.W. 211.94, 0.4447 mole, Aldrich 316393) was added in portions over about ten minutes. The slurry was allowed to warm to ambient temperature (about 20° C.) over two hours, then held at ambient temperature for about eighteen hours. Aqueous sodium hydroxide (484 g of 2.5N (10% by weight, F.W. 40.0, 1.21 mole), was added. The mixture was stirred for about thirty minutes.

The agitation was stopped the phases left to separate for three days at ambient temperature under nitrogen. The mixture was poured into a separatory funnel, liquid was decanted from the crystals in the toluene layer. Crystals were rinsed with a little toluene, then dried 30 minutes in a vacuum oven under nitrogen at about 35° C. oven temperature. Yield=16.5 g crystals.

Isolation from the liquid layers was done as follows:

The toluene layer was extracted once with aqueous sodium hydroxide (20 g of 10% aq. NaOH in about 200 ml water), the liquid layers were separated and the organic layer extracted once with water (about 100 ml). The combined aqueous portions were extracted once with toluene (about 50 ml), separated and the combined toluene portions were extracted once with water (about 200 ml). The toluene layer was concentrated in a one liter one neck round-bottom flask at reduced pressure (water aspirator vacuum), and maintained at a temperature below 100° C. during the concentration. The non-volatile organic residue was dried to constant weight in a vacuum oven at 60° under a stream of nitrogen. Yield=69.8 g powder.

Purification (2-propanol procedure)

A mixture of 2-propanol (127 g) and 1-(2-phenethyl)-4-anilinopiperidine (112.48 g;

made from 83.38 g of 1-(2-phenethyl)-4-piperidone) is heated and stirred in a one liter round bottom flask immersed in a heated water bath. At a bath temperature of about 75° C. a clear brown solution is present. The bath is gradually cooled and at about 63° C. the solution remains homogeneous. Further cooling to a bath temperature of about 51° C. gives a precipitate which blocks the magnetic stir bar from spinning. The water bath is heated back to about 70° C. and the stir bar restarts to suspend the slurry. The suspended slurry is gradually cooled and is finally held at about 0° C. to 10° C. for about two hours. The solid is collected on a Buchner funnel using aspirator vacuum and the filter cake is rinsed twice with chilled (about −10° C.) 2-propanol, using approximately 25 g each rinse. The filter cake is pressed dry under vacuum and the wet cake (111.3 g) is dried at about 60° C. in a vacuum oven under nitrogen to give crystallized 1-(2-phenethyl)-4-anilinopiperidine (100.75 g, 87.6% yield).

Synthesis of 1-(2-phenethyl)-4-anilinopiperidine

| Experiment | 1 | 2 | 3 | 4 | 5 | 6 |
|---|---|---|---|---|---|---|
| Phenethyl | “As received” | Distilled | Distilled | Distilled | “As received” | Distilled |
| piperidone | 98.9 wt. % | 100.0 wt. % | 100.0 wt. % | 100.0 wt. % | 97.2 wt. % | 100.0 wt. % |
| quality | | | | | | |
| Sodium | Aldrich | Aldrich | Made from | Aldrich | Aldrich | Aldrich |
| BH(OAc)₃ | | | NaBH₄ + | | | |
| source | | | HOAc | | | |
| grams | 64.2 | 64.2 | 69.2 | 64.3 | 64.2 | 83.4 |
| Phenethyl | | | | | | |
| piperidone | | | | | | |
| Sodium | 1.4 | 1.4 | 1.4(NaBH₄) | 1.4 | 1.4 | 1.3 |
| BH(OAc)₃ | | | | | | |
| equivalents | | | | | | |
| Aniline | 1.02 | 1.01 | 1.01 | 1.01 | 1.04 | 1.02 |
| equivalents | | | | | | |
| HOAc | 1.02 | 1.01 | 1.02 | 1.01 | 1.04 | 1.02 |
| equivalents | | | | | | |
| grams | 340 | 360 | 280 | 340 | 340 | 480 |
| toluene | | | | | | |
| Oven- | 87.9 | 88.7 | 94.7 | 86.3 | 87.8 | 112.7 |
| dried crude | | | | | | |
| grams | | | | | | |
| Contained | 83.2 | 84.4 | 86.0 | 82.9 | 81.1 | 108.0* |
| PAP | | | | | | |
| grams | | | | | | |
| Normalized | 95.9 | 95.1 | 94.2 | 96.6 | 95.1 | 96.8 |
| HPLC area | | | | | | |
| % PAP | | | | | | |
| Percent | 95.0 | 95.1 | 90.2 | 93.5 | 94.1 | 93.9* |
| Contained | | | | | | |
| Yield | | | | | | |

*estimated

All journal references and patent publications cited in this application are hereby incorporated by reference in their entirety.

What is claimed:

1. A process of preparing 1-(2-phenethyl)-4-anilinopiperidine, said process comprising:
reacting 1-(2-phenethyl)-4-piperidone with aniline in an aromatic solvent;
adding sodium triacetoxyborohydride or
sodium triacyloxyborohydride generated in-situ with an acid and sodium borohydride, to produce 1-(2-phenethyl)-4-anilinopiperidine;
subsequently isolating 1-(2-phenethyl)-4-anilinopiperidine.

2. A process of preparing 1-(2-phenethyl)-4-anilinopiperidine, said process comprising:
adding 1-(2-phenethyl)-4-piperidone with aniline in an aromatic solvent to sodium triacetoxyborohydride or
adding 1-(2-phenethyl)-4-piperidone with aniline in an aromatic solvent to sodium triacetoxyborohydride generated in-situ with acetic acid and sodium borohydride;
to produce 1 -(2-phenethyl)-4-anilinopiperidine;
subsequently isolating 1-(2-phenethyl)-4-anilinopiperidine.

3. The process according to claims 1 or 2 wherein:
the aromatic solvent is chosen from xylene, cumene, ethylbenzene, benzene, trimethyl benzene, ethyl toluene, cymene and toluene or the mixtures thereof optionally with an acid.

4. The process according to claim 3 wherein:
the aromatic solvent toluene optionally with an acid chosen from acetic, propionic, butyric, valeric, hexanoic, isobutyric, isovaleric, pivalic, 2-methylbutyric and benzoic acid.

5. The process according to claim 4 wherein:
the acid is acetic acid.

6. A process for purification of 1-(2-phenethyl)-4-anilinopiperidine by recrystallization from a solvent chosen from 1-propanol, 2-butanol, methyl isopropyl ketone, 2,2-dimethyl-1,3-dioxolane, tetrahydrofuran, 2-methyltetrahydrofuran, tetrahydropyran, 1,2-dimethoxyethane, 2-butanone, pyrrolidine, 1-methylpyrrolidine, triethylamine, diisopropylamine, acetonitrile, 2,2-dimethoxypropane, diethoxymethane, 1,3-dioxolane, 2-methyl-1,3-dioxolane, ethyl acetate, isopropyl acetate, ethyl isobutyrate and 2-propanol said process comprising,
providing 1-(2-phenethyl)-4-anilinopiperidine in the solvent;
isolating the 1-(2-phenethyl)-4-anilinopiperidine product.

7. The process according to claim 6 wherein:
the solvent is chosen from tetrahydrofuran, 2-methyltetrahydrofuran, tetrahydropyran, 1,2-dimethoxyethane, 2-butanone, pyrrolidine, 1-methylpyrrolidine, triethylamine, diethoxymethane, 1,3-dioxolane, 2-methyl-1,3-dioxolane, isopropyl acetate and 2-propanol.

8. The process according to claim 7 wherein the solvent is 2-propanol.
